(19) United States
(12) Patent Application Publication
(10) Pub. No.: US 2024/0226121 A1
(43) Pub. Date: Jul. 11, 2024

(54) PHYTOCANNABINOID FORMULATIONS AND METHODS FOR EXTRACTION

(71) Applicant: Pedro P. Perez, Miami, FL (US)

(72) Inventor: Pedro P. Perez, Miami, FL (US)

(21) Appl. No.: 18/433,084

(22) Filed: Feb. 5, 2024

Related U.S. Application Data
(63) Continuation-in-part of application No. 17/070,047, filed on Oct. 14, 2020, now abandoned.
(60) Provisional application No. 62/914,853, filed on Oct. 14, 2019.

(51) Int. Cl. A61K 31/00; A61K 9/00; A61K 9/06; A61K 9/20; A61K 9/48; A61K 47/12; A61K 47/22; A61K 47/24; A61K 47/36; A61K 47/44; A61K 47/46

(52) U.S. Cl. CPC A61K 31/658; A61K 9/0095; A61K 9/06; A61K 9/2009; A61K 9/2013; A61K 9/2018; A61K 9/2027; A61K 9/2054; A61K 9/2068; A61K 9/4858; A61K 9/4875; A61K 47/12; A61K 47/22; A61K 47/24; A61K 47/36; A61K 47/44; A61K 47/46

(57) ABSTRACT

A phytocannabinoid formulation comprises an amount of at least about 90 percent by weight of one or more phytocannabinoid. The phytocannabinoid formulation further comprises an amount of at least about 0.1 percent by weight of a plurality of lipophilic molecules including at least one saturated straight chain C₁₄-C₂₀ fatty acid and at least one unsaturated ω-6 C₁₈-C₂₂ fatty acid. The phytocannabinoid formulation also includes an amount of at least about 0.1 percent by weight of at least one bioflavonoid.

### CLAIM OF PRIORITY

The present application is a Continuation-In-Part of and claims priority to a previously filed U.S. Non-Provisional patent application, namely, that having Ser. No. 17/070,047, and a filing date of Oct. 14, 2020, and further claims priority to a previously filed U.S. Provisional patent application, namely, that having Ser. No. 62/914,853 and a filing date of Oct. 14, 2019, with the contents of both prior applications being incorporated herein by reference in their entireties.

### BACKGROUND OF THE INVENTION

### Field of the Invention

The present invention is directed to phytocanabinoid formulations, as well methods for extraction of phytocannabinoids.

### Description of the Related Art

Phytocannabinoids are a class of diverse chemical compounds that act on cannabinoid receptors on cells that modulate physiological responses in the brain, peripheral nervous and immune systems. Native endocannabinoid ligands are produced naturally in the body by humans and animals. Naturally occurring phytocannabinoids are found in cannabis and some other plants, and synthetic phytocannabinoids may be manufactured chemically, and they bind to receptors throughout the body and control downstream signal transduction. One example of a phytocannabinoid is cannabidiol (“CBD”), which is a major substituent in hemp and hemp extracts. Some researchers believe that CBD shows promise of potential clinical applications in a variety of medical conditions. It may have multiple potential applications such as for the treatment of epilepsy and other motor disorders, inflammation, mood and anxiety disorders, sleep dysfunction and eating disorders. CBD is also considered a promising antineoplastic agent on the basis of its in vitro and in vivo activity against tumor cells.

The endocannabinoid system regulates many physiological processes involved in relaxation, eating, sleeping, certain inflammatory responses and even cognitive function. There are two types of cannabinoid receptors found throughout the body, CB1 and CB2, but they are most abundant in the brain and immune system respectively. In fact, the CB1 receptor is the most densely populated G-coupled protein receptor in the human brain. New evidence indicates that a cannabinoid-like ligands act on wide variety of biological targets, such as the transient receptor potential cation channel, nuclear receptors and other orphaned G-coupled protein receptors, i.e., TRPV1, PPAR, GPR18 and GPR55, and represents a fascinating area to develop new therapeutic targets.

CB1 receptors are found primarily in the brain, more specifically in the basal ganglia and in the limbic system, including the hippocampus. They are also found in the cerebellum and in both male and female reproductive systems. CB1 receptors are absent in the medulla oblongata, the part of the brain stem responsible for respiratory and cardiovascular functions. Thus, there is not the risk of respiratory or cardiovascular failure that can be produced by some drugs, such as opioids. CB1 receptors appear to be responsible for the euphoric and anticonvulsive effects of cannabis.

CB2 receptors are predominantly found in the immune system or immune-derived cells with the greatest density in the spleen. While found only in the peripheral nervous system, a report does indicate that CB2 is expressed by a subpopulation of microglia in the human cerebellum. CB2 receptors appear to be responsible for the anti-inflammatory and possibly other therapeutic effects of phytocannabinoids. CBD binds only weakly to both the CB1 and CB2 receptor sites and its health benefits cannot be explained with traditional cannabinoid receptor binding. It is unclear how CBD functions and its mechanism of action is a current topic of investigation.

### SUMMARY OF THE INVENTION

In view of the numerous and substantial benefits to be realized from phytocannabinoid formulations, it is one aspect of the present invention to provide one or more phytocannabinoid formulation comprising at least one phytocannabinoid component. It is another aspect of the present invention to provide phytocannabinoid formulations comprising a plurality of phytocannabinoid components.

It is a further aspect of the present invention to provide one or more methods for the extraction of the phytocannabinoids disclosed herein.

In at least one embodiment, a phytocannabinoid formulation in accordance with the present invention comprises an amount of at least about 90 percent by weight of at least one phytocannabinoid, an amount of at least about 0.1 percent by weight of a plurality of lipophilic molecules including at least one saturated straight chain C₁₈-C₃₄ fatty alcohols and at least one saturated straight C₁₄-C₂₄ fatty acids and at least one unsaturated ω-3 C₁₈-C₂₄ fatty acids, ω-6 C₁₈-C₂₂ fatty acids, ω-7 C₁₈-C₂₀ fatty acids, and ω-9 C₁₈-C₂₀ fatty acids, and an amount of at least about 0.1 percent by weight of at least one bioflavonoid. In at least one further embodiment, a phytocannabinoid formulation comprises an amount of at least about 90 percent by weight of a plurality of phytocannabinoids. The plurality of phytocannabinoids may include, but are in no manner limited to, cannabidiol, cannabidivarin, cannabigerol, cannabichromene, cannabinol, cannabidiolic acid, and delta-9-tetrahydrocannabinol.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a photographic illustration of the morphology of PC12 cells treated with various CBD formulations.

FIG. 2 is a graphical representation of neurite outgrowth in PC12 cells treated with various CBD formulations.

FIG. 3 is a graphical representation of neuronal cell survival in PC12 cells treated with various CBD formulations.

FIG. 4 is a graphical representation of neurite outgrowth in PC12 cells treated with various nerve growth factor and CBD formulations.

FIG. 5 is a graphical representation of neuronal cell survival in PC12 cells treated with various nerve growth factor and CBD formulations.

FIG. 6 is a graphical representation of anti-inflammatory potential in PC12 cells treated with various CBD formulations.

### DETAILED DESCRIPTION

The present invention is directed to phytocannabinoid formulations, as well as methods for the extraction of various phytocannabinoid formulations.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, and animals. The present invention is not limited by the illustrated ordering of acts or events, as some acts may occur in different orders and/or concurrently with other acts or events. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting of the invention. As used herein, the indefinite articles “a”, “an” and “the” should be understood to include plural reference unless the context clearly indicates otherwise. Further, the singular shall include the plural and the plural shall include the singular, unless specifically stated otherwise.

The phrase “and/or”, as used herein, should be understood to mean “either or both” of the elements so conjoined, i.e., elements that are conjunctively present in some cases and disjunctively present in other cases.

As used herein, “or” shall have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of.”

As used herein, the terms “including”, “includes”, “having”, “has”, “with”, or variants thereof, are intended to be inclusive similar to the term “comprising.”

The term “phytocannabinoid” as used herein shall mean any of the one hundred plus naturally occurring cannabinoids produced in the trichomes of the cannabis plant and includes, but is in no means limited to, cannabidiol, cannabidivarin, cannabigerol, cannabichromene, cannabinol, cannabidiolic acid, and delta-9-tetrahydrocannabinol. When derived from the plant and consumed, phytocannabinoids interact with receptors in the human body to produce numerous psychotropic and therapeutic effects.

The term “phytocannabinoid formulation” as used herein shall mean any preparation comprising at least one phytocannabinoid in combination with at least one additional component, as disclosed in detail hereinafter.

The term “lipophilic molecules” as used herein refers to molecular compounds that are readily dissolve in fats, oils, lipids and certain non-polar solvents. Lipophilic molecules may be derived from natural waxes including, but not limited to, sugar cane wax, rice bran wax, carnauba wax, candelilla wax, japan wax, ouricury wax, bayberry wax, shellac wax, sunflower wax, orange wax, and beeswax. The lipophilic molecules extracted from natural waxes include, but are not limited to, palmitic acid, linoleic acid, linolenic acid, oleic acid, and steric acid.

The term “bioflavonoids” as used herein are flavonoids which are biologically active in the human body including, but shall not be limited to, rutin, naringin, hesperidin, neohesperidin, neohesperidin dihydrochalcone, naringenin, hersperitin, nomilin, and gallic acid, among other bioflavonoids well known in the art.

The term “nanoparticles” refers to particles having a d90 ranging from about 1 to about 900 nm and preferably from about 1 to about 500 nm.

The term “microparticles” refers to particles having a d90 ranging from about 1 to about 90 μm and preferably from about 1 to about 50 μm.

A “nanoemulsion” as used herein refers to an emulsion composed of nanoscale droplets of an immiscible liquid dispersed within another.

As previously stated, in at least one aspect, the present invention is directed to a phytocannabinoid formulation. In at least one embodiment of the present invention, a phytocannabinoid formulation comprises an amount of least one phytocannabinoid, and in one further embodiment, a phytocannabinoid formulation comprises an amount of a plurality of phytocannabinoids.

As before, one or more phytocannabinoids may be selected from any of the one hundred plus naturally occurring cannabinoids produced in the trichomes of the cannabis plant and includes, but is in no means limited to, cannabidiol, cannabidivarin, cannabigerol, cannabichromene, cannabinol, cannabidiolic acid, and delta-9-tetrahydrocannabinol.

In at least one embodiment of the present invention, a phytocannabinoid formulation comprises an amount of at least about 90% by weight of at least one phytocannabinoid. In at least one further embodiment, a phytocannabinoid formulation comprises an amount of at least about 90% by weight of a plurality of phytocannabinoids.

According to another embodiment, a phytocannabinoid formulation includes about 1 to about 50 mg of a plurality of phytocannabinoids.

In accordance with one embodiment of the present invention, a phytochemical formulation comprises cannabidiol in an amount of about 40 percent to about 75 percent by weight. In another embodiment, the present phytocannabinoid formulation comprises cannabidivarin in an amount of about 0.02 percent to about 2 percent by weight. In one further embodiment, a phytocanabinoid formulation includes an amount of cannabigerol in an amount of about 0.5 percent to about 5 percent by weight. In yet another embodiment, a phytocannabinoid formulation comprises cannabichromene in an amount of about 0.5 percent to about 7 percent by weight. A further embodiment of the present phytocannabinoid formulation includes cannabinol in an amount of about 0.04 percent to about 2 percent by weight. Still another embodiment of a phytocannabinoid formulation in accordance with the present invention comprises an amount of cannabidiolic acid of about 0.05 percent to about 0.5 percent by weight. In yet one further embodiment, a phytochemical formulation comprises delta-9-tetrahydrocannabinol in an amount of about 0.01 percent to about 0.3 percent by weight.

In accordance with another embodiment of the present invention, a phytochemical formulation comprises cannabidiol in an amount of about 40 percent to about 75 percent by weight, cannabidivarin in an amount of about 0.02 percent to about 2 percent by weight, an amount of cannabigerol in an amount of about 0.5 percent to about 5 percent by weight, cannabichromene in an amount of about 0.5 percent to about 7 percent by weight, cannabinol in an amount of about 0.04 percent to about 2 percent by weight, an amount of cannabidiolic acid of about 0.05 percent to about 0.5 percent by weight, and delta-9-tetrahydrocannabinol in an amount of about 0.01 percent to about 0.3 percent by weight.

In one further embodiment, a phytocannabinoid formation in accordance with the present invention comprises an amount of lipophilic molecules. In one embodiment, the lipophilic molecules comprise at least one straight chain C₁₈-C₃₄ fatty alcohols and at least one saturated straight C₁₄-C₂₄ fatty acids and at least one unsaturated ω-3 C₁₈-C₂₄ fatty acids, ω-6 C₁₈-C₂₂ fatty acids, ω-7 C₁₈-C₂₀ fatty acids, and ω-9 C₁₈-C₂₀ fatty acids, and in at least one further embodiment, the phytocannabinoid formulation comprises an amount of lipophilic molecules having a plurality of saturated straight chain C₁₈-C₃₄ fatty alcohols and at least one saturated straight C₁₄-C₂₄ fatty acids and at least one unsaturated ω-3 C₁₈-C₂₄ fatty acids, ω-6 C₁₈-C₂₂ fatty acids, ω-7 C₁₈-C₂₀ fatty acids, and ω-9 C₁₈-C₂₀ fatty acids. In still one further embodiment, the amount of lipophilic molecule comprises at least one unsaturated ω-3 C₁₈-C₂₄ fatty acids, ω-6 C₁₈-C₂₂ fatty acids, ω-7C₁₈-C₂₀ fatty acids, and ω-9 C₁₈-C₂₀, and in yet one other embodiment, the phytocannabinoid formulation comprises a plurality of lipophilic molecules comprising a plurality of unsaturated ω-6 C18-C22 fatty acids.

A phytocannabinoid formulation in accordance with at least one embodiment of the present invention includes an amount of lipophilic molecules comprising at least one saturated straight chain C₁₈-C₃₄ fatty alcohols and at least one saturated straight C₁₄-C₂₄ fatty acids and at least one unsaturated ω-3 C₁₈-C₂₄ fatty acids, ω-6 C₁₈-C₂₂ fatty acids, ω-7 C₁₈-C₂₀ fatty acids, and ω-9 C₁₈-C₂₀ fatty acids.

In accordance with the present invention, lipophilic molecules included into phytocannabinoid formulations include, but are in no manner limited to, molecules derived from natural waxes such as, by way of example only, sugar cane wax, rice bran wax, carnauba wax, candelilla wax, japan wax, ouricury wax, bayberry wax, shellac wax, sunflower wax, orange wax, and beeswax. In at least one further embodiment, lipophilic molecules extracted from natural waxes include, but are not limited to, palmitic acid, linoleic acid, linolenic acid, oleic acid, and steric acid.

In accordance with at least one embodiment of the present invention, a phytocannabinoid formulation in accordance with the present invention comprises at least about 0.1% by weight of at least one lipophilic molecule, and in another embodiment, the phytocannabinoid formulation comprises at least about 0.1% by weight of a plurality of lipophilic molecules. In one embodiment, a phytocannabinoid formulation in accordance with the present invention comprises at amount of at least about 0.1 percent by weight of a plurality of lipophilic molecules including at least one saturated straight chain C₁₈-C₃₄ fatty alcohols and at least one saturated straight C₁₄-C₂₄ fatty acids and at least one unsaturated ω-3 C₁₈-C₂₄ fatty acids, ω-6 C₁₈-C₂₂ fatty acids, ω-7 C₁₈-C₂₀ fatty acids, and ω-9 C₁₈-C₂₀ fatty acids.

At least one further embodiment of phytocannabinoid formulation in accordance with the present invention comprises about 0.1% by weight of lipophilic molecules including one or more saturated straight chain C₁₈-C₃₄ fatty alcohols and at least one saturated straight C₁₄-C₂₄ fatty acids and at least one unsaturated ω-3 C₁₈-C₂₄ fatty acids, ω-6 C₁₈-C₂₂ fatty acids, ω-7 C₁₈-C₂₀ fatty acids, and ω-9 C₁₈-C₂₀ fatty acids.

Suitable lipophilic molecules include, but are not limited to, those derived from natural waxes such as, but is not limited to, sugar cane wax, rice bran wax, carnauba wax, candelilla wax, japan wax, ouricury wax, bayberry wax, shellac wax, sunflower wax, orange wax, and beeswax. Suitable lipophilic molecules extracted from natural waxes include, but are not limited to, palmitic acid, linoleic acid, linolenic acid, oleic acid, and steric acid.

According to one embodiment of the present invention, a phytocannabinoids formulation includes the following lipophilic molecules in the weight percentages stated below, which are based on 100% of the total weight of lipophilic molecules in the phytocannabinoid formulation:
about 0.01-0.3% (by weight) palmitic acid,
about 0.01-2.0% linoleic acid,
about 0.01-0.6% alpha linolenic acid,
about 0.01-0.4% oleic acid,
about 0.1-0.8% steric acid,
about 0.01-0.09% arachidic acid,
about 0.01-0.09% heneicosanoic acid,
about 0.1-0.9% behenic acid,
about 0.1-0.9% tricosanoic acid,
about 0.01-0.9% lignoceric acid,
about 0.05-0.9% cerotic acid,
about 0.1-1.0% heptacosanoic acid,
about 0.05-1.5% montanic acid,
about 0.2-2.6% melissic acid,
about 0.05-1.6% docosahexaenoic acid,
about 0.05-0.9% docosapentaenoic acid,
about 0.05-1.9% docosatetraenoic acid,
about 0.05-0.9% docosadienoic acid,
about 0.01-1.8% erucic acid,
about 0.01-0.09% nervonic acid,
about 0.01-8.0% cetyl alcohol-hexadecanol-palmityl alcohol,
about 0.01-5.0% 1-heptadecanol,
about 0.01-1.0% 1-eicosanol-arachidyl alcohol,
about 0.01-3.0% 1-docosanol-behenyl alcohol,
about 1.0-15.0% lignoceryl alcohol-1-tetracosanol,
about 1.0-12.0% 1-hexacosanol-ceryl alcohol,
about 0.01-2.0% 1-heptacosanol,
about 0.5-20.0% 1-octacosanol,
about 15.0-40.0% 1-triacontanol-melissyl alcohol,
about 10.0-20.0% dotriacontanol, and
about 5.0-15.0% tetratriacontanol.

According to one embodiment, a phytocannabinoids formulation includes phytocannabinoids and lipophilic molecules in a weight ratio from about 20:1 to about 1000:1. According to one further embodiment, the weight ratio of phytocannabinoids to lipophilic molecules is about 100:1 to about 1000:1.

According to another embodiment, a phytocannabinoids formulation includes about 90% by weight of a phytocannabinoids and about 0.1% by weight of lipophilic molecules based upon 100% total weight of the a phytocannabinoids formulation. In one further embodiment, a phytocannabinoids formulation includes about 90% to about 99% by weight of a phytocannabinoids and about 0.1% to about 5% by weight of lipophilic molecules.

A phytocannabinoid formulation in accordance with one further aspect of the present invention comprises an amount of least one bioflavonoid, and in one further embodiment, a phytocannabinoid formulation comprises an amount of a plurality of bioflavonoids.

The plurality of bioflavonoids which may be incorporated into a phytocannabinoid formulation in accordance with the present invention include, but are in no manner limited to, rutin, naringin, hesperidin, neohesperidin, neohesperidin dihydrochalcone, naringenin, hersperitin, nomilin, and gallic acid.

In at least one embodiment of the present invention, a phytocannabinoid formulation comprises an amount of at least about 0.1% by weight of at least one bioflavonoid. In at least one further embodiment, a phytocannabinoid formulation comprises an amount of at least about 0.1% by weight of a plurality of bioflavonoid.

According to one embodiment of the present invention, a phytocannabinoid formulation includes the following bioflavonoids in the amounts stated below, by weight:
about 20-120 units rutin,
about 25-100 units naringin,
about 7000-20000 units hesperidin,
about 5-100 units neohesperidin,
about 10-100 units neohesperidin dihydrochalcone,
about 5-100 units naringenin,
about 5-100 units hersperitin,
about 50-150 units nomilin, and
about 120,000-1,000,000 units gallic acid.

According to one further embodiment, a phytocannabinoid formulation includes the following bioflavonoids in the amounts stated below:
7 about 20-120 ppm rutin,
about 25-100 ppm naringin,
about 7000-20000 ppm hesperidin,
about 5-100 ppm neohesperidin,
about 10-100 ppm neohesperidin dihydrochalcone,
about 5-100 ppm naringenin,
about 5-100 ppm hersperitin,
about 50-150 ppm nomilin, and
about 120-1000 mg/g gallic acid.

According to yet one further embodiment, a phytocannabinoid formulation includes about 90% by weight of phytocannabinoids and about 0.1% to about 5% by weight of lipophilic molecules, and about 0.1% to about 5% by weight of bioflavonoids.

A phytocannabinoid formulation in accordance with the present invention may be incorporated into any of a plurality of delivery systems. As one example, a phytocannabinoid formulation may be provided in the form of an oral dosage, such as beads, pellets, granules, capsules, soft or hard, sachets, tablets, powders, dispersible powders capable of effervescing upon addition of water, aqueous or oily suspensions, emulsions, syrups, elixirs, or lozenges. Additional examples of an oral dosage delivery systems include, but are not limited to, a suspension in an aqueous or non-aqueous liquid solution, or an emulsion which can be a soft drink, tea, milk, coffee, juice, sports drink, or water.

As such, a phytocannabinoid formulation in accordance with the present invention may include one or more excipients or additives. Suitable excipients and additives include, but are not limited to, additional antioxidants, inert diluents, such as lactose, sodium carbonate, calcium phosphate, and calcium carbonates, granulating and disintegrating agents, such as corn starch and algenic acid, binders, such as starch, lubricants, such as magnesium stearate, stearic acid and talc, preservatives, such as ethyl or propyl p-hydroxybenzoate, colorants, flavoring agents, release modifying agents, thickeners, and any combination of any of the foregoing. Suitable antioxidants include, but are not limited to, bioflavonoids, flavonoids, flavonols, flavanones, flavones, flavonals, flavanolols, and flavanols.

Suitable inert solid diluents include, but are not limited to, calcium carbonate, calcium phosphate and kaolin. Suitable diluents for soft capsules include, but are not limited to, water and oils such as peanut oil, liquid paraffin, corn oil, wheat germ oil, soybean oil, and olive oil.

Aqueous suspensions or dispersions contain the active ingredient, for example, in fine powder form together with one or more suspension or dispersion (or wetting) agents. Suitable suspension agents include, but are not limited to, sodium carboxymethylcellulose, methylcellulose, hydroxypropylmethylcellulose, sodium alginate, polyvinyl-pyrrolidone, gum tragacanth and gum acacia. Suitable dispersing or wetting agents include, but are not limited to, lecithin, condensation products of an alkylene oxide with fatty acids, condensation products of ethylene oxide with long chain aliphatic alcohols, condensation products of ethylene oxide with partial esters derived from fatty acids and a hexitol such as polyoxyethylene sorbitol monooleate, or condensation products of ethylene oxide with partial esters derived from fatty acids and hexitol anhydrides.

Oily suspensions may be formulated by suspending the active ingredient in a vegetable oil or a mineral oil. The oily suspensions may also contain a thickening agent such as carnauba wax, candelilla wax, rice bran wax, beeswax, hard paraffin, or cetyl alcohol.

Dispersible powders and granules suitable for preparation of an aqueous suspension by the addition of water contain the active ingredient, for example, together with a dispersing agent, wetting agent, or suspending agent. Suitable dispersing agents, wetting agents, and suspending agents include those mentioned above.

A phytocannabinoid formulation may be provided in the form of an oil-in-water emulsion. The oily phase may be a vegetable based oil or a mineral based oil. Suitable emulsifying agents include, for example, naturally occurring gums such as acacia and tragacanth gum, naturally occurring phosphatides such as soy bean, lecithin, esters and partial esters derived from fatty acids and hexitol anhydrides and condensation products of partial esters with ethylene oxide, such as polyoxyethylene sorbitan monooleate.

Syrups and elixirs may be formulated with sweetening agents such as glycerol, propylene glycol, sorbitol, aspartame, or sucrose, and may also contain a demulcent, preservative, flavoring, or coloring agent.

A phytocannabinoid formulation in accordance with one embodiment of the present invention comprises a delivery system which provides for controlled release of phytocannabinoids, lipophilic molecules, and bioflavonoids, for example, to provide effective doses of phytocannabinoids over extended periods of time. At least one embodiment of the present invention is a phytocannabinoid formulation in a controlled release dosage form, such as a solid dosage form, containing about 1 to about 50 mg of a plurality of phytocannabinoids, about 1 to 100 mg of lipophilic molecules, and 1 to 500 mg of bioflavonoids. For example, the controlled release dosage form may release about 10% to about 35% by weight of the total phytocannabinoids, lipophilic molecules, and bioflavonoids within about 2 hours in an in vitro dissolution test, and about 40% to about 70% by weight of the total phytocannabinoids, lipophilic molecules, and bioflavonoids within about 8 hours.

Solid controlled release dosage forms can be coated so as to further prolong the release of the phytocannabinoids into the gastrointestinal tract, or to prevent the release of the phytocannabinoids in the stomach in order to prevent or attenuate the gastrointestinal side effects which can accompany phytocannabinoids released into the stomach.

A phytocannabinoid formulation in accordance with at least one embodiment of the present invention is administered orally to a mammal, e.g., a human being, but it may also be administered by other routes of administration, such as intravenously or subcutaneously.

The following are but a few examples of the types of preparations which may be realized utilizing a phytocannabinoid formation in accordance with the present invention.

### Example 1: Tablet Composition

| Component | Amount, weight percent |
|---|---|
| phytocannabinoid formulation | 10 |
| vitamin C formulation | 10 |
| lactose | 30 |
| microcrystalline cellulose | 32 |
| sodium citrate | 8 |
| croscarmellose sodium | 2 |
| sodium lauryl sulphate | 0.5 |
| polyacrylin potassium | 3 |
| talc | 3 |
| silicon dioxide | 0.25 |
| magnesium stearate | 1 |
| candelilla wax | 0.25 |

### Example 2: Gelatin Capsules Composition

| Component | Amount, weight percent |
|---|---|
| phytocannabinoid formulation | 2 |
| vitamin C formulation | 2 |
| rice powder | 93 |
| magnesium stearate | 3 |

### Example 3: Softgel Composition I

| Component | Amount, weight percent |
|---|---|
| phytocannabinoid formulation | 3.33 |
| vitamin C formulation | 3.33 |
| olive oil | 86.74 |
| soy lecithin | 6.6 |

### Example 4: Softgel Composition II

| Component | Amount, weight percent |
|---|---|
| phytocannabinoid formulation | 3.33 |
| vitamin C formulation | 3.33 |
| olive oil | 51.3 |
| Wheat germ oil | 25.6 |
| rosemary oil | 3.33 |
| bee Pollen, power | 13.3 |

### Example 5: Time Release Tablet Composition

| Component | Amount, weight percent |
|---|---|
| phytocannabinoid formulation | 10 |
| vitamin C formulation | 10 |
| ethyl cellulose | 26 |
| microcrystalline cellulose | 17 |
| hydroxypropyl ethylcellulose | 18 |
| stearic acid | 18 |
| magnesium stearate | 1 |

### Example 6: Beverages Composition

Healthy beverages such as, but not limited to, juices, soft drinks, tea, milk, coffee, sports drinks and water may be homogenized with a phytocannabinoid formulation and/or a vitamin C formulation wherein a measured cup of the resulting beverage contain about 5 milligrams to about 50 milligrams of the phytocannabinoid formulation and/or of the phytocannabinoid formulation and a vitamin C formulation.

### Example 7: Foodstuff Composition

A wide variety of foodstuffs may be homogenized with a phytocannabinoid formulation in accordance with the present invention wherein; the resulting foodstuffs contain about 5 milligrams to about 50 milligrams of the phytocannabinoid formulation and/or of the phytocannabinoid formulation and a vitamin C formulation, per a single serving size specified for the particular foodstuff.

A vitamin C formulation in accordance with at least one embodiment of the present invention, as referenced above in Examples 1 through 7, and below with regard to the formulation of CBD2, comprises at least about 90% by weight of vitamin C, at least about 0.1% by weight of lipophilic molecules comprising one or more saturated straight C₁₈-C₃₄ fatty alcohols, one or more saturated straight C₁₄-C₂₄ fatty acids, and one or more unsaturated ω-3 C₁₈-C₂₄ fatty acids, ω-6 C₁₈-C₂₂ fatty acids, ω-7 C₁₈-C₂₀ fatty acids, ω-9 C₁₈-C₂₀ fatty acids, and at least about 0.1% by weight of bioflavonoids, based upon 100% total weight of the vitamin C formulation.

A phytocannabinoid formulation in accordance with at least one embodiment of the present invention, as referenced above in Examples 1 through 7, and below with regard to the formulation of CBD2, comprises at least about 90% by weight of phytocannabinoids, at least about 0.1% by weight of lipophilic molecules comprising (i) one or more saturated straight C₁₈-C₃₄ fatty alcohols one or more saturated straight C₁₄-C₃₀ fatty acids and (ii) one or more unsaturated ω-3 C₁₈-C₂₄ fatty acids, ω-6 C₁₈-C₂₂ fatty acids, ω-7 C₁₈-C₂₀ fatty acids, ω-9 C₁₈-C₂₀ fatty acids and at least about 0.1% by weight of bioflavonoids, based upon 100% total weight of the phytocannabinoids preparation. Furthermore, the phytocannabinoids in the preceding phytocannabinoid formulation comprises cannabidiol (“CBD”) in an amount of about 40 percent to about 75 percent by weight, cannabidivarin (“CBDV”) in an amount of about 0.02 percent to about 2 percent by weight, an amount of cannabigerol (“CBG”) in an amount of about 0.5 percent to about 5 percent by weight, cannabichromene (“CBC”) in an amount of about 0.5 percent to about 7 percent by weight, cannabinol (“CBN”) in an amount of about 0.04 percent to about 2 percent by weight, an amount of cannabidiolic acid (“CBDA”) of about 0.05 percent to about 0.5 percent by weight, and delta-9-tetrahydrocannabinol (“D9-THC”) in an amount of about 0.01 percent to about 0.3 percent by weight.

The following is one exemplary phytocannabinoid formulation prepared in accordance with the present invention, which is designated at CBD1.

EXEMPLARY PHYTOCANNABINOID
FORMULATION - CBD1:

| Component | Amount, weight percent |
|---|---|
| phytocanabinoid formulation | 3 |
| maltodextrin | 79 |
| fiber blend | 10 |
| vegetable oil blend | 6 |
| sodium benzoate | 0.5 |
| potassium sorbate | 0.5 |
| citric acid | 1 |

The phytocannabinoid formulation in CBD1 comprises CBD 0.31%, CBDV 0.02%, CBG 0.01%, CBC 0.01%, CBN 0.04%, CBDA 0.03%, and D9-THC 0.01%.

2 The following is one exemplary phytocannabinoid formulation prepared in accordance with the present invention, which is designated at CBD2.

EXEMPLARY PHYTOCANNABINOID
FORMULATION - CBD2:

| Component | Amount, weight percent |
|---|---|
| phytocannabinoid formulation | 3 |
| vitamin C formulation | 10 |
| maltodextrin | 69 |
| fiber blend | 10 |
| vegetable oil blend | 6 |
| sodium benzoate | 0.5 |
| potassium sorbate | 0.5 |
| citric acid | 1 |

The phytocannabinoid formulation in CBD2 comprises CBD 0.31%, CBDV 0.02%, CBG 0.01%, CBC 0.01%, CBN 0.04%, CBDA 0.03%, and D9-THC 0.01%.

The following is a phytocannabinoid formulation prepared for testing, discussed below, which is designated at CBD3.

PHYTOCANNABINOID FORMULATION - CBD3:

| Component | Amount, weight percent |
|---|---|
| Phytocannabinoids, nanoemulsion | 3 |
| maltodextrin | 79 |
| fiber blend | 10 |
| vegetable oil blend | 6 |
| sodium benzoate | 0.5 |
| potassium sorbate | 0.5 |
| citric acid | 1 |

The phytocannabinoids in the nanoemulsion of CBD3 comprises CBD 0.3%, CBDV 0.02%, CBG 0.05%, CBC 0.12%, CBN (ND), CBDA (ND), and D9-THC (ND), wherein (ND) is non-detectable.

The following is a phytocannabinoid formulation prepared for testing, discussed below, which is designated at CBD4.

PHYTOCANNABINOID FORMULATION - CBD4:

| Component | Amount, weight percent |
|---|---|
| Phytocannabinoids, liposomal emulsion | 3 |
| maltodextrin | 79 |
| fiber blend | 10 |
| vegetable oil blend | 6 |
| sodium benzoate | 0.5 |
| potassium sorbate | 0.5 |
| citric acid | 1 |

The phytocannabinoids in the liposomal emulsion of CBD4 comprises CBD 0.45%, CBDV (ND), CBG 0.01%, CBC 0.02%, CBN (ND), CBDA (ND), and D9-THC (ND), wherein (ND) is non-detectable.

The following is a phytocannabinoid formulation prepared for testing, discussed below, which is designated at CBD5.

PHYTOCANNABINOID FORMULATION - CBD5:

| Component | Amount, weight percent |
|---|---|
| phytocannabinoids, Bioperine emulsion | 3 |
| maltodextrin | 79 |
| fiber blend | 10 |
| vegetable oil blend | 6 |
| sodium benzoate | 0.5 |
| potassium Sorbate | 0.5 |
| citric acid | 1 |

The phytocannabinoids in the Bioperene emulsion of CBD5 comprises CBD 0.88%, CBDV 0.02%, CBG 0.01%, CBC 0.02%, CBN (ND), CBDA (ND), and D9-THC (ND), wherein (ND) is non-detectable.

The foregoing formulations for CBD1 through CBD5 were utilized in tests were conducted on PC12 rat neuronal cells to determine which formulation most effectively delivers the known neurological health benefits associated with phytocannabinoids. In order to model neuronal activities such and nerve formation, regeneration, repair and survival, PC12 rat neuronal cells were cultured in serum-free conditions and either untreated or treated with nerve growth factor (“NGF”) or various formulations of CBD over a five day period. During this five day period both neurite outgrowth and viability were measured. The CBD1 and CBD2 formulations both outperformed the CBD3, CBD4, and CBD5 formulations by as much as 75% on days 3 and 5 post treatment. For example, CBD1 and CBD2 show 20% and 35% more neurites respectively on day three than CBD5, and 32% and 40% more activity respectively on day five. These data are statistically significant with 95% confidence and when CBD1 and CBD2 are compared for neurite outgrowth activity as much as a 75% increase is seen compared to CBD3 and CBD4. Further, trypan blue exclusion showed that CBD1 and CBD2 increase neuronal viability by 45% and 57% respectively over CBD5 by day five of serum starvation. Again, these data are statistically significant at a 95% confidence and a similarly significant increase in viability was also seen with CBD1 and CBD2 when compared to CBD3 and CBD4. Cannabidiol is known to act on TRKA receptors on PC12 cells as demonstrated by the ability of the tyrosine kinase inhibitor K252a to block NGF and CBD induced neurite outgrowth. Here we confirm that CBD1 and CBD2 act through the TRKA NGF receptor system to stimulate neurite outgrowth and show for the first time that CBD binding to the TRKA receptor promotes neuronal cell viability and survival. It is also interesting to note the CBD2 contains vitamin C, which also has neurotrophic effects through non-TRKA mechanisms. While the data do not quite show a statistically significant difference, CBD2 activity is less affected by K252a than CBD1, suggesting that the benefits of CBD2 are due to the combined physiological effect of cannabidiol and vitamin C through two different mechanisms. Taken together these data show that CBD1 and CBD2 are the best formulations for the neurological health benefits of CBD. Moreover, these data show a new activity of CBD in promoting neuronal cell viability, which has important implications for the use of CBD in the repair of damaged nerves and stem cell therapy. Further, TRKA signaling is associated with anti-inflammatory responses, and CBD1 signals through TRKA have a greater effect on cell attachment as well and therefore also anti-inflammatory potential. Lastly, combinatorial effects of vitamin C can enhance and boost the health benefit of CBD by acting through a second mechanism to support neuronal and immune system health.

### Materials and Methods

Cells and reagents: PC12 cells were maintained in DMEM containing 7.5% heat inactivated horse serum and 7.5% fetal bovine serum (Fisher Scientific, Inc.) and cultured in T-75 vented Nunc brand tissue culture flasks (Fisher Scientific, Inc.). For passage, PC12 cells were rinsed with serum free DMEM and treated for 5-10 minutes with Trypsin. Cells were then collected, suspended in culture medium and plated at a 30% confluence. The PC12 cells were maintained between a 30% and 90% confluence in a CO₂ water-jacketed incubator maintained at 37° Celsius. Murine 2.5 S nerve growth factor (“NFG”) was purchased from Calbiochem, Inc. and dissolved in DMEM to a stock concentration of 100 mg/ml. K252a was purchased from Sigma Chemical Co. and dissolved in DMSO to a stock concentration of 1 mg/ml. CBD formulations were emulsified into DMEM at a concentration of 1 mg/ml.

Neurite outgrowth and serum-free survival assays: For both neurite outgrowth assays and serum-free survival assays, PC12 cells were collected and rinsed free of serum by centrifuging approximately 6×10 6 cells (a 90% confluent T-75 flask) pouring out the supernate and suspending the cells in DMEM and repeating this three times. For the final suspension the cells were placed in 1.0 ml DMEM and 10 μl of the cell suspension was counted on a hemocytometer. Cells were then diluted to 5×105 cells/ml of DMEM and 0.5 ml was seeded in the wells of a 24 well tissue culture plate (Fisher Scientific Inc.). At the time of cell seeding, for appropriate cells, NGF was added at a final 100 ng/ml concentration, K252a was added to appropriate wells at a final concentration of 100 nM and CBD was added to appropriate wells at a final test concentration of 10 μM. Cells were immediately and at various times tested for viability by removing them by rinsing from test wells and treating the cells with Trypan Blue and inspected at 10× magnification on a hemocytometer. The medium collected prior to rinsing was also collected and cells remaining attached to the wells were counted as a percentage of attached cells and then removed and combined with all cells from the well to count total cell viability. The percent viability was determined as the number of cells in a count of 100 that excluded the Trypan Blue. These cells were then discarded. To measure viability cells were then collected on days 1, 3 and 5 to yield viability counts on days 0, 1, 3, and 5. Wells for assessment of neurite outgrowth were visually inspected and assessed for the percent of cells having a neurite extending at least one cell diameter. After assessment, these cells were returned to the incubator to continue the formation of neurite. The time course experiments were done in duplicate. Cells treated with K252a were separate experiments assessed on either day 3 or 5 and were done in triplicate.

### Results

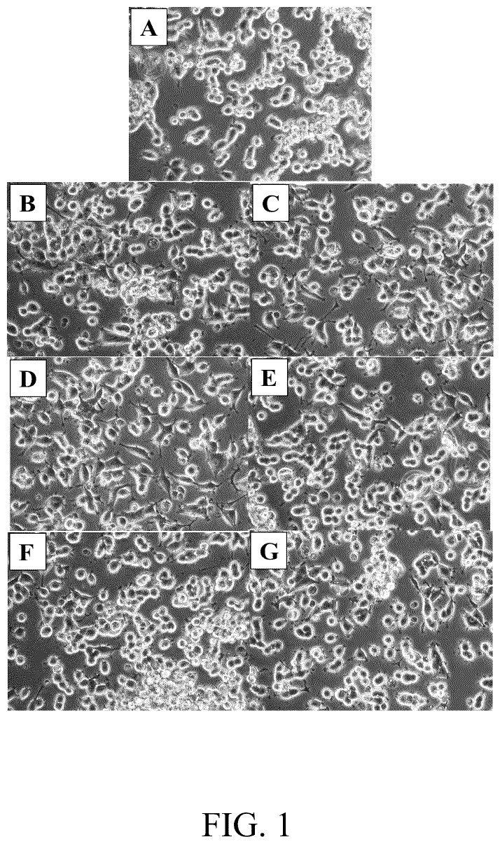

FIG. 1 is a photographic illustration of the morphology of PC12 cells treated with various CBD formulations, namely, the CBD formulations designated as CBD1 through CBD5 as disclosed hereinabove.

PC12 cells were seeded on tissue culture plastic in serum free medium and at the time of cell seeding. Cells were photomicrographed on day three of culture at a total magnification of 320×, and the results are present in FIG. 1 wherein: (A) is illustrative of untreated cells; (B) is illustrative of cells treated with 100 ng/ml of nerve growth factor (NGF); (C) is illustrative of cells treated with 10 μM of CBD1; (D) is illustrative of cells treated with CBD2; (E) is illustrative of cells treated with CBD3; (D) is illustrative of cells treated with CBD4; and, (E) is illustrative of cells treated with CBD5.

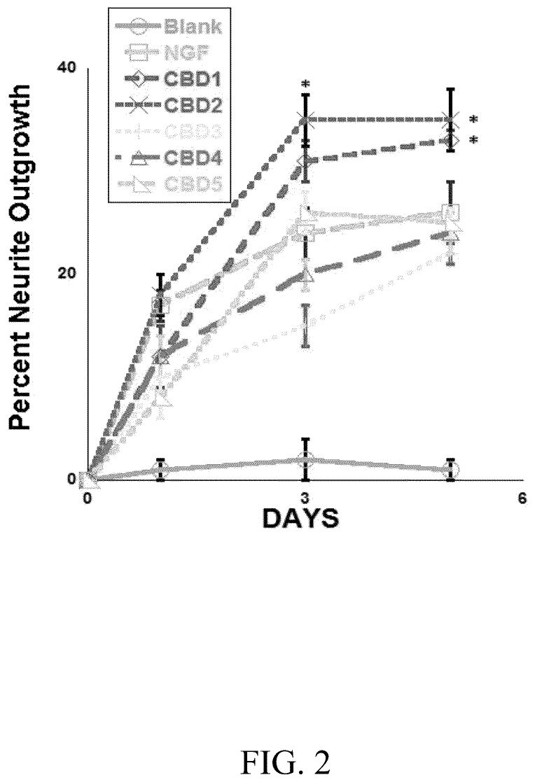

FIG. 2 is a graphical representation of neurite outgrowth in PC12 cells treated with various CBD formulations, namely, the CBD formulations designated as CBD1 through CBD5 as disclosed hereinabove.

As is readily seen from FIG. 2, cells treated with CBD1 and CBD2 are the most neuritogenic formulations of the CBD formulation tested. PC12 cells were seeded on tissue culture plastic in a serum free medium, and the percentage of cells that formed neuritis were counted by visual inspection over a five day period. Cells were either untreated, i.e., Blank, treated with 100 ng/ml nerve growth factor (NGF), or treated with 10 μM of one of the five different CBD formulations described above, namely, CBD1 through CBD5. CBD1 and CBD2 exhibited statistically significantly greater neuritogenic at 95% confidence (*) on days three and five (p<0.05, t-test) compared to the other CBD formulations.

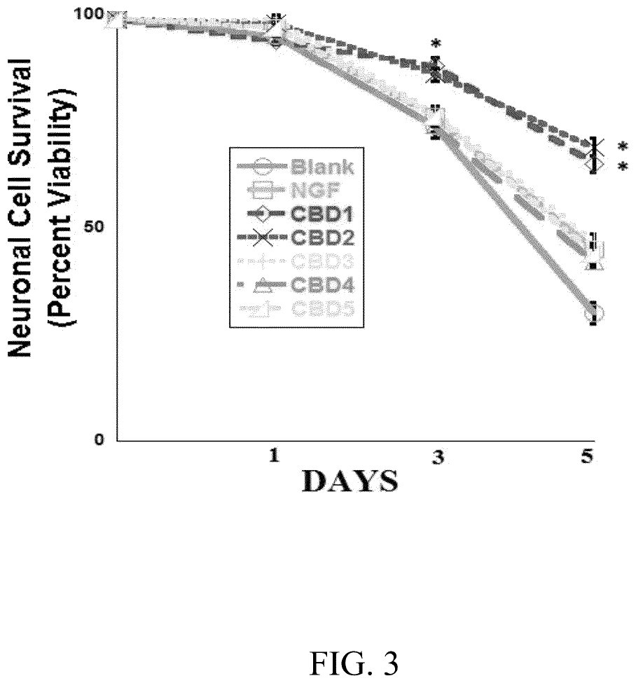

FIG. 3 is a graphical representation of neuronal cell survival in PC12 cells treated with various CBD formulations, namely, the CBD formulations designated as CBD1 through CBD5 as disclosed hereinabove.

As is readily seen from FIG. 3, cells treated with CBD1 and CBD2 are the most effective CBD formulations tested to promote neuronal survival. PC12 cells were seeded on tissue culture plastic in a serum free medium and cell viability was measured by the percentage of cells that exhibited trypan exclusion. Cells were either untreated, i.e., Blank, treated with 100 ng/ml nerve growth factor (NGF), or treated with 10 μM of one of the five different CBD formulations described above, namely, CBD1 through CBD5. As above, cells treated with CBD1 and CBD2 exhibited statistically significantly more viable on day five of serum starvation at 95% confidence (*) (p<0.05, t-test) compared to any of the other CBD formulations.

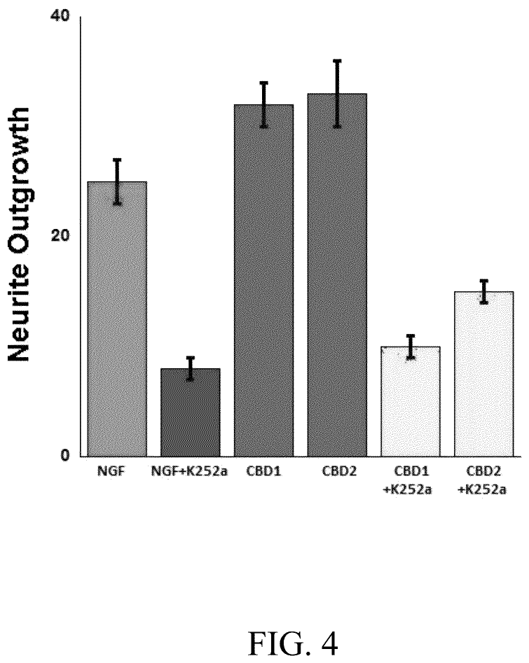

FIG. 4 is a graphical representation of neurite outgrowth in PC12 cells treated with various nerve growth factor and CBD formulations, namely, the CBD formulations designated as CBD1 and CBD2 as disclosed hereinabove.

Cannabidiol stimulates neurite outgrowth through the nerve growth factor TRKA tyrosine kinase receptor. PC12 cells were treated with either 100 ng/ml NGF or 10 μM of CBD1 or CBD2, and neurite outgrowth was assessed on day three of treatment. As maybe seen from FIG. 4, when the TRKA tyrosine kinase inhibitor, K252a, is concomitantly added at 0.1 μM to cells treated with CBD1 and CBD2, respectively, induced neurite outgrowth was inhibited.

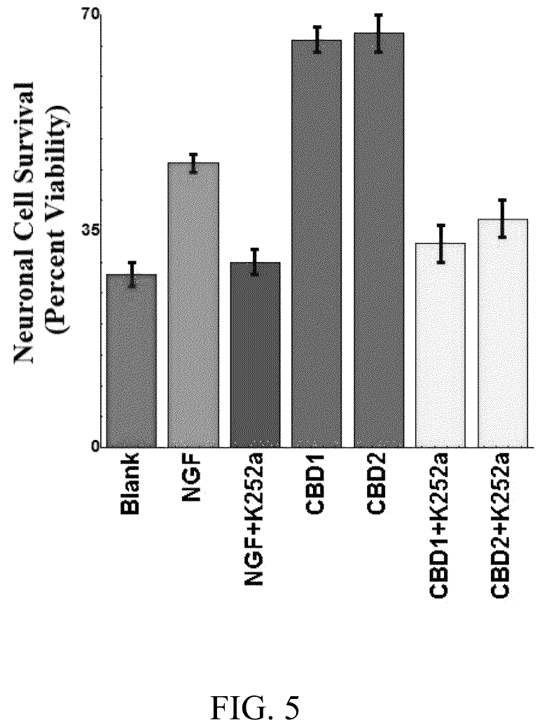

FIG. 5 is a graphical representation of neuronal cell survival in PC12 cells treated with various nerve growth factor and CBD formulations, namely, the CBD formulations designated as CBD1 and CBD2 as disclosed hereinabove.

Cannabidiol enhances neuronal cell survival by acting through the NGF TRKA tyrosine kinase receptor. PC12 cells were treated with either 100 ng/ml NGF or 10 μM of CBD1 or CBD2, and neuronal cell viability was measured on day five of treatment. The TRKA tyrosine kinase inhibitor, K252a when concomitantly added at 0.1 μM inhibited both CBD1 and CBD2 induced neuronal cell survival.

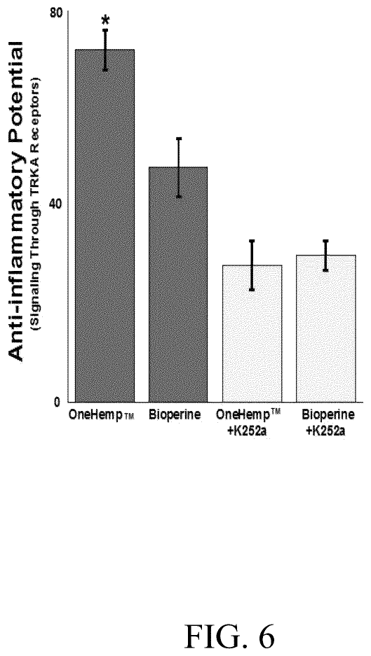

FIG. 6 is a graphical representation of anti-inflammatory potential in PC12 cells treated with various CBD formulations, namely, the CBD formulations designated as CBD1 through CBD5 as disclosed hereinabove.

CBD1, i.e., OneHemp, has greater anti-inflammatory TRKA signaling than Bioperine. Cells were cultured in the presence of 10 μM CBD1 or CBD5, i.e., Bioperine CBD, with and without 0.1 μM of the TRKA inhibitor, K252a, for two days in a serum-free DMEM. After three days, the percent of cells attached were measured and changes due to TRKA inhibition with K252a are presented as anti-inflammatory potential. The asterisk denotes confidence at 95%.

As also noted above, the present invention is further directed to methods of extraction of the natural hemp varieties, such as, Cannabis sativa species and/or by blending selected natural hemp varieties to obtain at least one of the phytocannabinoids present in the natural hemp.

The method of extraction of the present invention sizing an amount of solid natural hemp material, such as by grinding, chopping, etc., to a particle mesh size of about 500 to 2,000 microns, for continuous or batch operations in a solid-liquid immersion type percolation extraction system, or to a particle mesh size of about 100 to about 425 microns for continuous or batch operations in a dispersed-solids extraction system.

After sizing the amount of natural hemp material, the sized natural hemp material is contacted with an amount of a first solvent. More in particular, one or more phytocannabinoids are selectively extracted from the sized natural hemp material with an amount of a first solvent. In accordance with one embodiment of the present invention, a first solvent may include acetone, ethanol, isopropyl-alcohol and others. Mixtures of the aforementioned solvents can also be used in the extraction process in accordance with the present invention. In at least one embodiment of the present method, the ratio of sized natural hemp material to solvent, by weight, is about 1:4 to about 1:10. In at least one further embodiment the ratio of sized natural hemp material to solvent, by weight, is about 1:8.

In accordance with at least one embodiment of the present method, the first solvent is at a temperature of about 55° Celsius to about 75° Celsius, and in one further embodiment, the first solvent is at a temperature of about 60° Celsius. The phytocannabinoid rich phase produced as a result of contacting the sized natural hemp material with an amount of a first solvent comprises one or more phytocannabinoids.

The yield, i.e., the percent recovery by weight of phytocannabinoids with respect to the weight of phytocannabinoids in the amount of sized natural hemp material, once again, such as Cannabis sativa species, and/or by blending of select natural hemp varieties, in the phytocannabinoid rich phase obtained via the present method is about 75% to about 95%, with purity of about 50% to about 90%. In one embodiment, a phytocannabinoid rich phase comprising one or more phytocannabinoids obtained via the present method has a melting point of about 70° Celsius to about 180° Celsius. The phytocannabinoids obtained via the repent method can be analyzed and assayed through gas chromatography or liquid chromatography.

In accordance with one embodiment of the present invention, the sized natural hemp material is contacted with a first solvent in a solid-liquid immersion type percolating extractor. In at least one embodiment, the contact time in the solid-liquid immersion type percolating extractor is about 30 minutes to about 120 minutes, and in one further embodiment, the contact time is about 30 minutes to about 90 minutes.

In another embodiment of the present invention, the sized natural hemp material is contacted with a first solvent in a dispersed-solids extractor. In at least one embodiment, the contact time in the dispersed-solids extractor is about 1 hour to about 4 hours. In at least one embodiment, the sized natural hemp material is contacted with a first solvent in a dispersed-solids extractor under agitation at a speed of about 100 to 500 revolution per minute, and in one further embodiment, at a speed of about 250 revolutions per minute.

During an extraction process in accordance with the present invention, the natural mixture of phytocannabinoids becomes solubilized in the phytocannabinoid rich phase, in the form of a waxy residue. The waxy residue may be removed from the extractor in a variety of ways. In the case of a solid-liquid immersion type percolating extractor, a screw conveyer may be utilized to transfer the phytocannabinoid rich phase. In the case of the dispersed-solids extraction system, the phytocannabinoid rich phase may be transferred via pumping.

In accordance with at least embodiment of the present invention, the phytocannabinoid rich phase is contacted with an amount of a second solvent to form a purified phytocannabinoid rich phase, wherein the purified phytocannabinoid rich phase comprises the one or more phytocannabinoid present in the phytocannabinoid rich phase. In accordance with one embodiment of the present invention, the second solvent may comprise hexane, heptane, acetone, ethanol, and/or isopropyl-alcohol, among others.

In at least one embodiment of the present method, the purified phytocannabinoid rich phase comprises one or more phytocannabinoids is transferred to a crystallization unit, where the temperature of the purified phytocannabinoid rich phase is reduced to about −5° Celsius to about −25° Celsius to form crystals of the purified phytocannabinoid rich phase comprising one or more phytocannabinoids. In at least one embodiment, the crystallization unit comprises low speed agitation. The crystallized phytocannabinoid rich phase comprising one or phytocannabinoids, in at least one embodiment, is further processed in a centrifuge, after which, the crystallized phytocannabinoid rich phase is transferred to a dryer system, such as, by way of example only, an air dryer or vacuum dryer, in order to obtain a final processed phytocannabinoid rich phase comprising one or more phytocannabinoids.

The dried oil phytocannabinoid rich phase comprising one or more phytocannabinoids may be further purified, using various purification techniques including, but not limited to, dissolution, filtration, molecular distiller, crystallization, centrifugation and drying specially designed systems.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A phytocannabinoid formulation comprising:
an amount of at least about 90 percent by weight of at least one phytocannabinoid,
an amount of at least about 0.1 percent by weight of a plurality of lipophilic molecules including at least one saturated straight chain C₁₈-C₃₄ fatty alcohol and at least one saturated straight C₁₄-C₂₄ fatty acid, and at least one unsaturated ω-3 C₁₈-C₂₄ fatty acid, ω-6 C₁₈-C₂₂ fatty acid, ω-7 C₁₈-C₂₀ fatty acid, and ω-9 C₁₈-C₂₀ fatty acid,
an amount of at least about 0.1 percent by weight of at least one bioflavonoid,
wherein said phytocannabinoid formulation comprises about 1 to about 50 mg of the at least one phytocannabinoid,
wherein said phytocannabinoid formulation comprises about 1 to about 100 mg of the plurality of lipophilic molecules, and
wherein said phytocannabinoid formulation comprises about 1 to about 500 mg of the at least one bioflavonoid.

2. The phytocannabinoid formulation as recited in claim 1 comprising an amount of at least about 90 percent by weight of a plurality of phytocannabinoids.

3. The phytocannabinoid formulation as recited in claim 2 where the plurality of phytocannabinoids are selected from the group consisting of cannabidiol, cannabidivarin, cannabigerol, cannabichromene, cannabinol, cannabidiolic acid, and delta-9-tetrahydrocannabinol.

4. The phytocannabinoid formulation as recited in claim 1 wherein said formulation comprises about 40 percent to about 75 percent by weight of cannabidiol.

5. The phytocannabinoid formulation as recited in claim 1 wherein said formulation comprises about 0.02 percent to about 2 percent by weight of cannabidivarin.

6. The phytocannabinoid formulation as recited in claim 1 wherein said formulation comprises about 0.5 percent to about 5 percent by weight of cannabigerol.

7. The phytocannabinoid formulation as recited in claim 1 wherein said formulation comprises about 0.5 percent to about 7 percent by weight of cannabichromene.

8. The phytocannabinoid formulation as recited in claim 1 wherein said formulation comprises about 0.04 percent to about 2 percent by weight of cannabinol.

9. The phytocannabinoid formulation as recited in claim 1 wherein said formulation comprises about 0.5 percent to about 5 percent by weight of cannabidiolic acid.

10. The phytocannabinoid formulation as recited in claim 1 wherein said formulation comprises about 0.01 percent to about 0.3 percent by weight of delta-9-tetrahydrocannabinol.

11. The phytocannabinoid formulation as recited in claim 1 wherein the plurality of lipophilic molecules are selected from the group consisting of palmitic acid, linoleic acid, linolenic acid, oleic acid, and steric acid.

12. The phytocannabinoid formulation as recited in claim 1 wherein the at least one bioflavinoid is selected from the group consisting of rutin, naringin, hesperidin, neohesperidin, neohesperidin dihydrochalcone, naringenin, hersperitin, nomilin, and gallic acid.

13. A phytochemical formulation comprising:
cannabidiol in an amount of about 40 percent to about 75 percent by weight,
cannabidivarin in an amount of about 0.02 percent to about 2 percent by weight,
cannabigerol in an amount of about 0.5 percent to about 5 percent by weight,
cannabichromene in an amount of about 0.5 percent to about 7 percent by weight,
cannabinol in an amount of about 0.04 percent to about 2 percent by weight,
cannabidiolic acid in an amount of about 0.05 percent to about 0.5 percent by weight,
delta-9-tetrahydrocannabinol in an amount of about 0.01 percent to about 0.3 percent by weight,
an amount of at least about 0.1 percent by weight of a plurality of lipophilic molecules including at least one saturated straight chain C₁₈-C₃₄ fatty alcohol and at least one saturated straight C₁₄-C₂₄ fatty acid, and at least one unsaturated ω-3 C₁₈-C₂₄ fatty acid, ω-6 C₁₈-C₂₂ fatty acid, ω-7 C₁₈-C₂₀ fatty acid, and ω-9 C₁₈-C₂₀ fatty acid,
an amount of at least about 0.1 percent by weight of at least one bioflavonoid,
wherein said phytochemical formulation comprises the cannabidiol, the cannabidivarin, the cannabigerol, the cannabichromene, the cannabinol, and the delta-9-tetrahydrocannabinol in the total combined amount of about 1 to about 50 mg,
wherein said phytochemical formulation comprises about 1 to about 100 mg of the plurality of lipophilic molecules, and
wherein said phytochemical formulation comprises about 1 to about 500 mg of the at least one bioflavonoid.
